(19) United States
(12) Patent Application Publication
(10) Pub. No.: US 2015/0234980 A1
(43) Pub. Date: Aug. 20, 2015

(54) GENOME SEQUENCE SYSTEMS AND METHODS

(71) Applicant: Tin Lap LEE, Shatin (HK)

(72) Inventor: Tin Lap LEE, Shatin (HK)

(21) Appl. No.: 14/184,293

(22) Filed: Feb. 19, 2014

(51) Int. Cl. G06F 19/18; C12Q 1/68

(52) U.S. Cl. CPC G06F 19/18; C12Q 1/6869

(57) ABSTRACT

According to embodiments of the invention, systems and methods are provided for capturing nucleotide sequence and providing associated modification snapshots with timestamps. The system and/or method are adapted to receive a sample containing a genome sequence from an individual or an organism (hereinafter collectively referred to as a “subject”) that uses the genome for encoding DNA or RNA. The genome sequence is then identified and stored in a database, for access at a later time. Multiple samples from multiple individuals may be taken, analyzed, and compare via the provided invention. Moreover, genetic as well as non-genetic information may be determined and stored in the database. Such information may include data relating to phenotypic features and clinical biological data.

### FIELD OF THE INVENTION

This invention is generally related to biotechnology and bioinformatics. Specifically, this invention is related to capturing nucleotide sequence and providing associated modification snapshots with timestamps.

### BACKGROUND OF THE INVENTION

Genome sequencing involves the process of determining a complete DNA sequence of an organism's genome at a single time. Genome sequencing is the sequencing or all of an organisms chromosomal DNA or mitochondrial DNA (in plants). Testing and determining of genome sequences has become a fairly well-known and relatively simple process. Only a small sample is need from a subject in order to determine genome sequence.

By itself, a single sample providing a single genome sequence does not provide any important information. However, numerous genome sequences containing numerous samples that have been taken over time may be able to tell us something about the subject providing the sample.

Thus, in view of the foregoing, there is a need for a device, system and method for determining and storing genome sequences of one or more subjects over a given time interval.

### SUMMARY OF THE INVENTION

According to embodiments of the invention, systems and methods are provided for capturing nucleotide sequence and providing associated modification snapshots with timestamps. The system and/or method are adapted to receive a sample containing a genome sequence from an individual or an organism (hereinafter collectively referred to as a “subject”) that uses the genome for encoding DNA or RNA. The genome sequence is then identified and stored in a database, for access at a later time. Multiple samples from multiple individuals may be taken, analyzed, and compare via the provided invention.

In an embodiment of the disclosed invention, a genome sequence machine that inserts DNA and DNA modification snapshots with timestamps is provided. The machine's components may include: a device configured to identify genomes sequences, a processor, a database, and a memory. The memory may store instructions that cause the processor to execute a method.

The method begins by receiving a sample containing a genome sequence from an individual or a subject that uses the genome as a primary genomic material. A genome sequence is then identified from the sample. The method proceeds by associating a timestamp with the identified genome sequence, wherein the timestamp is associated with a time at which the sample is received. The next step involves processing the identified genome sequence. The processing may include storing the genome sequence to the database.

Next, the method proceeds by receiving a new sample containing a new genome sequence from the subject sampled, and identifying the new genome sequence from the new sample. Next, a timestamp is associated with the new genome sequence. The timestamp is associated with a time at which the new sample is received. Next, the new genome sequence and the stored genome sequence are processed. The processing may include comparing the stored genome sequence with the new genome sequence. After the comparing is done, any changes are noted that have occurred with respect to the genome sequence of the subject

In a further embodiment, the method may also have a step of associating non-genetic information with the first identified genome sequence and the second identified genome sequence. The information may be a phenotypic feature or clinical biological information. The phenotypic information may be, for example, race, eye color, skin color, height, weight, and/or body size. The clinical biological information may be, for example, blood pressure or any other diagnostic. An additional step may be provided of comparing the non-genetic information associated with the first identified genome sequence to the information associated with the second identified genome sequence. Finally, any changes may be noted that have occurred with respect to the non-genetic information.

In other embodiments, the aforementioned described system and method may be used with respect to two different subjects. In these embodiments, two samples are taken, a first from a first subject, and a second form a second subject. The respective first genome sequence and second genome sequence are determined from the first sample and the second sample. The processing may involve comparing the first genome sequence to the second genome sequence to seek genetic patterns between the first genome sequence and the second genome sequence. After the comparing is completed, an analysis may be provided with respect to the genetic patterns involving the first genome sequence and the second genome sequence.

In yet another embodiment of the disclosed invention, a method is provided. The method employs the following steps. The first step is directed to providing a genome sequence machine device, wherein the genome sequence machine has a device configured to identify genome sequences, a processor, a database and a memory. The second step involves receiving a first sample containing a first genome sequence from a subject that uses the genome as a primary genomic material and identifying the first genome sequence. The third step is directed to associating a timestamp with the identified first genome sequence, wherein the timestamp is associated with a time of which the first sample is received. Then, data is associated with the identified first genome sequence, wherein the data relates to non-genetic information. The next step involves processing the identified first genome sequence, wherein the processing includes storing the first genome sequence to the database. At a later time, a second sample may be received containing a second genome sequence from the subject sampled. The second genome of the second sample may then be determined, and a corresponding timestamp may be associated. Information may be associated with the identified second genome sequence, wherein the information relates to non-genetic information.

The method may proceed by continuing to receive new samples of the subject and storing genome sequences identified from the new samples to the database, wherein each of the stored genome sequences is associated with a timestamp. The stored genomes may be processed. The processing may include identifying patterns from the stored genome sequences, wherein the patterns include genetic patterns and non-genetic patterns. The processing may also include computing rates of change of the patterns of the stored genome sequences using the timestamps associated with the stored genome sequences. Finally, the genome sequences in view of the timestamps associated with the stored genome sequences of the subject may be displayed on a display.

It is, therefore, an objective of the disclosed invention to provide a system and/or a method for identifying and storing genome sequence information from one or more subjects.

In accordance with these and other objects which will become apparent hereinafter, the invention will now be described with particular reference to the drawings.

### BRIEF DESCRIPTION OF THE DRAWINGS

FIG. 1 is a flow diagram of a genome sequence method according to an embodiment of the present invention.

FIG. 2 is a flow diagram of an exemplary Genome Snapshot Technology Implementation.

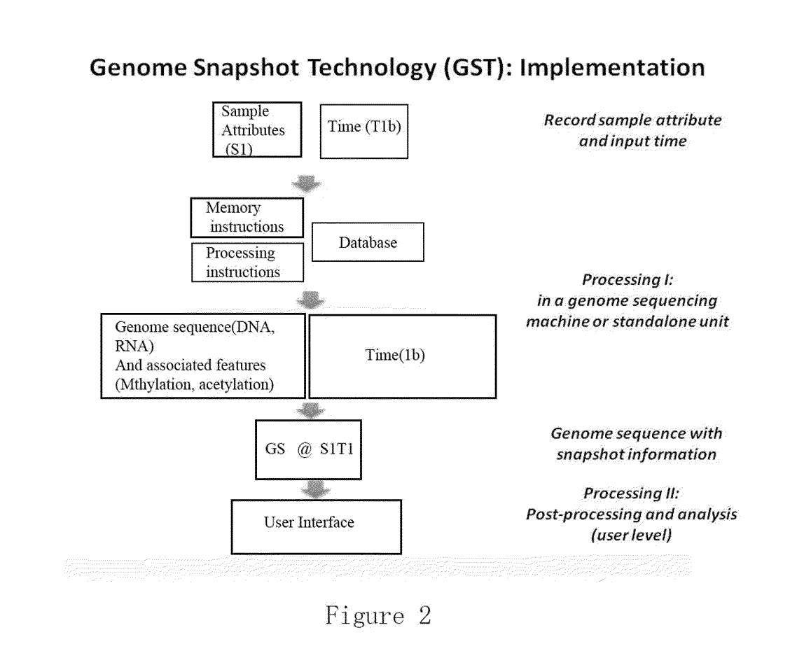

FIG. 3 is a block diagram of an exemplary Snapshot Databank.

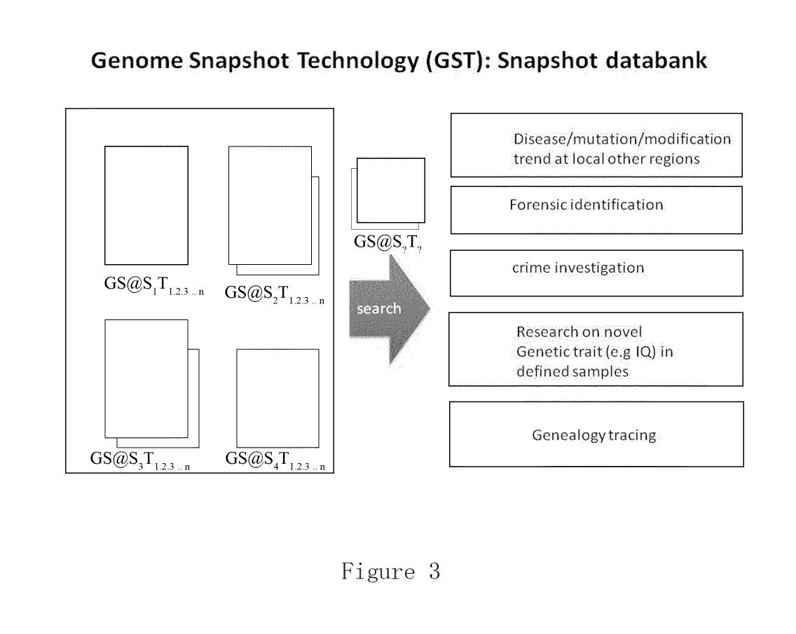

### DETAILED DESCRIPTION

The provided method and/or system involve a revolutionary process that takes and uploads DNA and DNA modification snapshots with timestamps for human and non-human organisms conveniently and quickly to a secured repository that can optionally protect privacy. The system will use this data for analysis, DNA & DNA modification recovery, preventive care, health forecast, wellness, research, data mining, and any other purposes that will make use of the data.

Under this invention, DNA sequencing and DNA modification are stored in different file formats such as FASTA, AB1, GFF, and SCF. The system can plug in different software modules in order to parse different DNA file formats, transform, and load into the data repository. Inventive features include backup & recovery features, in which a backup or the process of backing up refers to making copies of data so that these additional copies may be used to restore the original after a data loss event. Since the DNA repository contains DNA & DNA modification snapshots at different historic time, a doctor or research scientist or a computer module can be used to prescribe a chemical way to restore the current DNA of the living organism to a prior state.

As far as cryptography features, encryption is the process of transforming data using an algorithm to make it unreadable to anyone except those possessing the key. The system uses different algorithms to encrypt DNA sequence and modification data before storing it into the DNA repository. Other devices are used. Examples include computers, DNA sequencing devices, communication devices. A DNA sequencing device or computers with DNA files can use our proprietary communication device or software to send DNA files to the central DNA Snapshot Repository.

Referring now to the figures, systems and methods are provided for capturing nucleotide sequence and providing associated modification snapshots with timestamps. In one embodiment, a genome sequence machine is provided. The system is adapted to receive a sample containing a genome sequence from an individual or an organism (hereinafter collectively referred to as a “subject”) that uses the genome for encoding DNA or RNA; identifying the genome sequence; associating a timestamp with the identified genome sequence, wherein the timestamp is associated with a time of which the sample is received; processing the identified genome sequence and the associated timestamp, wherein the processing includes storing the genome sequence to the database; receiving a new sample containing a new genome sequence from the individual or the subject sampled at a later time; identifying the new genome sequence from the new sample; associating a timestamp with the new genome sequence, wherein the timestamp is associated with a time of which the new sample is received; and processing the new genome sequence and the stored genome sequence, wherein the processing includes comparing the stored genome sequence with the new genome sequence to look for differences between the stored genome sequence and the new genome sequence; and after the comparing is completed, noting that a change has occurred with respect to the genome sequence of the individual or subject, if the new genome sequence associated with the later timestamp varies from the stored genome sequence.

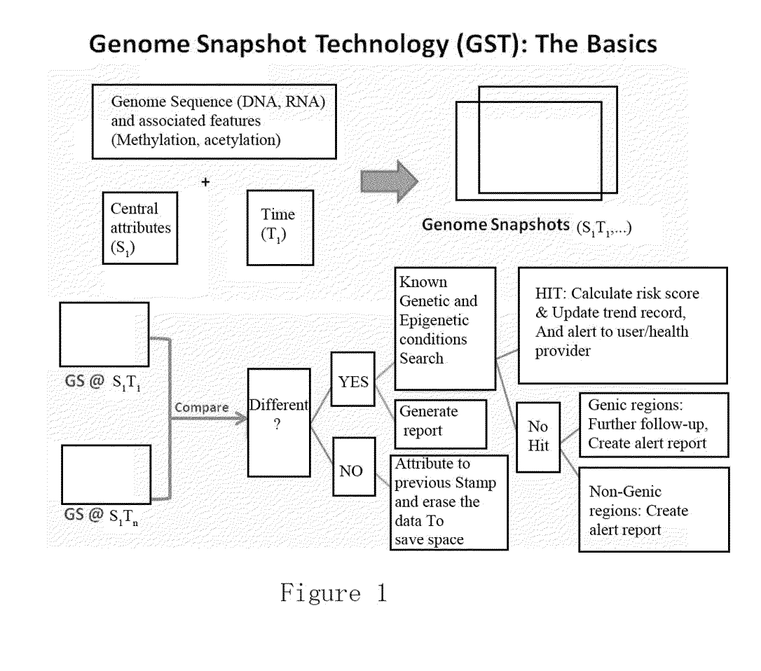

Referring now to FIG. 1, a sample containing a new genome sequence from an individual or a subject that uses the genome as a primary genomic material is captured as a genome snapshot at a time Tₙ. The sample can be used to compare with other past stored genome sequences of the same individual or subject (at T₁, as an example) to look for differences between the stored genome sequence and the new genome sequence. If attributes of the genome sequence is the same as the past stored genome sequences, it would be unnecessary to save the new sample so that such data can be erased. On the other hand, if the attributes are different, actions are taken to note for the differences, including searching for known genetic and epigenetic conditions. If a known condition is indeed found, then further actions can be taken to i) calculating a risk score and update a trend record, and ii) alerting to a provider can be performed. If no known conditions can be found, then further following up may be required, besides from creating alert reports.

The above method of observing variations of genome sequences of an individual or subject can be further facilitated by storing such information in files. Commonly, various file formats have been developed to store attributes regarding genome information. In one example, the Genome Variation Format (GVF) is a common standard simple file format used for describing alterations of genome sequences relative to one or more reference genomes. The Genome Variation Format (GVF), a file format for describing sequence variants at nucleotide resolution in relation to a referenced genome. GVF, similar to other file formats currently known in the art, is restricted by certain requirements and constraints per specification of the file format. For example, the GVF format has the same nine-column tab delimited format. Each column contains pragmas and attributes related to sequential information of the given genome. Because of such sequential arrangement, no information with respect to the time of the underlying genome data is required to be recorded as part of the file format. This causes problems when manipulation of non-sequential genome data may be required in certain circumstances, especially in systems where it would be unnecessary to record all genome data of the same individual or subject matter over time, where a majority of the genome information stays intact. As such, one feature of the present invention provides greater flexibility regarding the missing timing elements of current systems is to optionally include the timing information as part of the time format. Various timing information may be included. In one instance, the time of sampling can be added into the file of a relevant genome sample taken in a certain time, so that the file can be stored in the systems, while other non-relevant sample information can be selectively ignored. Advantageously, this enhances system efficiency.

While the disclosed invention has been taught with specific reference to the above embodiments, a person having ordinary skill in the art will recognize that changes can be made in form and detail without departing from the spirit and the scope of the invention. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the invention.

What is claimed is:

1. A genome sequence machine that inserts DNA and DNA modification snapshots with timestamps, comprising:
a device configured to identify genomes sequences;
a processor;
a database;
a memory storing instructions, wherein the instructions comprise:
receiving a sample containing a genome sequence from an individual or a subject that uses the genome as a primary genomic material;
identifying the genome sequence;
associating a timestamp with the identified genome sequence, wherein the timestamp is associated with a time at which the sample is received;
processing the identified genome sequence, wherein the processing includes:
storing the genome sequence to the database;
receiving a new sample containing a new genome sequence from the subject sampled;
identifying the new genome sequence from the new sample;
associating a timestamp with the new genome sequence, wherein the timestamp is associated with a time at which the new sample is received; and
processing the new genome sequence and the stored genome sequence, wherein the processing includes:
comparing the stored genome sequence with the new genome sequence; and
after the comparing is completed,
noting any changes that have occurred with respect to the genome sequence of the subject.

1. The genome sequence machine of claim 1, wherein the instructions further comprise:
associating non-genetic information with the first identified genome sequence and the second identified genome sequence, wherein the information comprises a phenotypic feature or clinical biological information;
comparing the non-genetic information associated with the first identified genome sequence to the information associated with the second identified genome sequence; and
noting any changes that have occurred with respect to the non-genetic information.

2. The genome sequence machine of claim 2, wherein the phenotypic feature is selected from the group consisting of:
race;
eye color;
skin color;
height;
weight; and
body size.

3. A genome sequence machine that inserts DNA and DNA modification snapshots with timestamps, comprising:
a device configured to identify genomes sequences;
a processor;
a database; and
a memory storing instructions, wherein the instructions comprise:
receiving a first sample containing a genome sequence from a first subject that uses the genome as a primary genomic material;
identifying the genome sequence of the first sample;
associating a first timestamp with the identified genome sequence of the first sample, wherein the second timestamp is associated with a time at which the first sample is received;
processing the identified genome sequence of the first sample, wherein the processing includes:
storing the genome sequence of the first sample to the database;
receiving a second sample containing a second genome sequence from a second subject, wherein the first subject is related to the second subject genetically;
identifying the second genome sequence from the second sample;
associating a second timestamp with the second genome sequence, wherein the timestamp is associated with a time at which the second sample is received;
processing the second genome sequence and the first genome sequence, wherein the processing includes:
comparing the first genome sequence to the second genome sequence to seek genetic patterns between the first genome sequence and the second genome sequence; and
after the comparing is completed, providing an analysis with respect to the genetic patterns involving the first genome sequence and the second genome sequence.

4. A method comprising:
providing a genome sequence machine device, wherein the genome sequence machine has a device configured to identify genome sequences, a processor, a database and a memory;
receiving a first sample containing a first genome sequence from a subject that uses the genome as a primary genomic material;
identifying the first genome sequence;
associating a timestamp with the identified first genome sequence, wherein the timestamp is associated with a time of which the first sample is received;
associating data with the identified first genome sequence, wherein the data relates to non-genetic information;
processing the identified first genome sequence, wherein the processing includes storing the first genome sequence to the database;
receiving a second sample containing a second genome sequence from the subject sampled at a later time;
identifying the second genome sequence from the second sample;
associating a timestamp with the second genome sequence, wherein the timestamp is associated with a time at which the new sample is received;
associating information with the identified second genome sequence, wherein the information relates to non-genetic information;
continuing to receive new samples of the subject and storing genome sequences identified from the new samples to the database, wherein each of the stored genome sequences is associated with a timestamp;
processing the stored genome sequences, wherein the processing includes:
identifying patterns from the stored genome sequences, wherein the patterns include genetic patterns and non-genetic patterns;
computing rates of change of the patterns of the stored genome sequences using the timestamps associated with the stored genome sequences;
displaying on a display the genome sequences in view of the timestamps associated with the stored genome sequences of the subject.

5. The method of claim 6, wherein the non-genetic information comprises a phenotypic feature, further wherein the phenotypic feature is selected from the group consisting of:
race;
eye color;
skin color;
height;
weight; and
body size.

6. The method of claim 6, wherein the non-genetic information comprises clinical biological information.

7. The method of claim 6, wherein the non-genetic information comprises blood pressure.

8. The method of claim 6, wherein the non-genetic information comprises a phenotypic feature.

9. The method of claim 6, wherein the non-genetic information comprises a feature relating to environmental feature.
